(19) United States
(12) Patent Application Publication
(10) Pub. No.: US 2018/0238860 A1
(43) Pub. Date: Aug. 23, 2018

(54) INDIVIDUAL METHOD PREDICTIVE OF THE DNA-BREAKING GENOTOXIC EFFECTS OF CHEMICAL OR BIOCHEMICAL AGENTS

(71) Applicants: NEOLYS DIAGNOSTICS, Lyon (FR); INSERM (INSTITUT NATIONAL DE LA SANTÉ ET DE LA RECHERCHE MÉDICALE), Paris (FR); CENTRE LEON BERARD, Lyon (FR); CENTRE NATIONAL DE LA RECHERCHE SCIENTIFIQUE, Paris Cedex 16 (FR); UNIVERSITE CLAUDE BERNARD LYON 1, Villeurbanne Cedex (FR)

(72) Inventors: Nicolas FORAY, Meyrie (FR); Mélanie FERLAZZO, Ugine (FR); Lauréne SONZOGNI, Bourg-en-Bresse (FR); Larry BODGI, Lyon (FR); Sandrine PEREIRA, Meyrargues (FR)

(21) Appl. No.: 15/751,815

(22) PCT Filed: Aug. 16, 2016
(86) PCT No.: PCT/FR2016/052083

§ 371 Date: Feb. 9, 2018

(30) Foreign Application Priority Data
Aug. 19, 2015 (FR) 1501745
Oct. 20, 2015 (FR) 1559962

(51) Int. Cl. G01N 33/50; G01N 33/68

(52) U.S. Cl. CPC G01N 33/5014; G01N 33/5044; G01N 2800/60; G01N 2800/709; G01N 2800/40; G01N 33/68

(57) ABSTRACT

A predictive method of cell toxicity after exposure to chemical elements breaking DNA directly or indirectly (particularly certain metals, pesticides and certain active substances for chemotherapy) and which is based on the determination and cross-checking of a plurality of cellular and enzymatic parameters and criteria.

### CROSS-REFERENCE TO RELATED APPLICATIONS

The present application is a National Stage Application of PCT International Application No. PCT/FR2016/052083 (filed on Aug. 16, 2016), under 35 U.S.C. § 371, which claims priority to French Patent Application Nos. 1501745 (filed on Aug. 19, 2015) and 1559962 (filed on Oct. 20, 2015), which are each hereby incorporated by reference in their respective entireties.

### TECHNICAL FIELD

The invention relates to the field of toxicology and more particularly the field of laboratory genotoxicological methods. The invention relates more particularly to a novel predictive method of cell toxicity after exposure to chemical elements breaking DNA directly or indirectly (particularly certain metals, pesticides and certain active substances for chemotherapy) and which is based on the determination and cross-checking of a plurality of cellular and enzymatic parameters and criteria.

### BACKGROUND

More and more numerous data in the literature demonstrate that double-strand breaks (DSBs) are the DNA damage best correlated with cell lethality and toxicity—if they are not repaired—and with genomic instability and with cancer risk—if they are poorly repaired (Jeggo and Lobrich, “DNA double-strand breaks: their cellular and clinical impact?”, Oncogene 26(56) p. 7717-7719 (2007); Joubert et al., “Radiation biology: major advances and perspectives for radiotherapy”, Cancer Radiotherapy 15(5) p. 348-354 (2011). Originally established for radiation-induced DSBs, such a conclusion appears to be valid for all DNA-breaking agents. As such, an evaluation of the toxic and carcinogenic risk based on the quantification of DSBs and the functionalities of the repair pathway thereof appears to be promising. However, the determination of DSBs and the repair and signaling models governing same are still far from resulting in consensus among radiobiologists and genotoxicologists. Conversely, some works have demonstrated a quantitative correlation between the number of non-repaired DSBs and the cellular radiosensitivity of human cells using the immunofluorescence technique, and have proposed a molecular model in conflict with the current paradigm (Joubert et al., “DNA double-strand break repair defects in syndromes associated with acute radiation response: at least two different assays to predict intrinsic radiosensitivity?”, Int. J. Radiation Biology 84(2), p. 1-19 (2008); Joubert, et al. (aforementioned article from 2011)). More recently, the same group of researchers demonstrated specific responses of certain human tissues to metals (particularly Pb, Cd, Al) (Viau et al., “Cadmium inhibits non-homologous end-joining and over-activates the MRE11-dependent repair pathway”, Mutation Research 654 p. 13-21 (2008); Gastaldo et al., “Lead contamination results in late and slowly repairable DNA double-strand breaks and impacts upon the ATM-dependent signaling pathways”, Toxicology Letters 173, p. 201-214 (2007); Gastaldo et al., “Induction and repair rate of DNA damage: A unified model for describing effects of external and internal irradiation and contamination with heavy metals”, J Theoretical Biology 251 p. 68-81 (2008)).

Toxicity and cancer may be the results of various external agents such as physical agents (X-rays, particles, UV, heat), chemical agents (alkylating agents, certain active substances used in chemotherapy, certain metals), biological agents (such as certain viruses or bacteria). Among these genotoxic stress factors, ionizing radiations are the external agent for which the biological effects are best documented (Thomas et al., “Impact of dose-rate on the low-dose hyper-radiosensitivity and induced radioresistance response”, International Journal of Radiation Biology, 89(10) p 813-822 (2013); Colin C. et al., “MRE11 and H2AX biomarkers in the response to low-dose exposure: balance between individual susceptibility to radiosensitivity and to genomic instability”, International Journal of Low Radiation: 8(2) p 96-106 (2011)); Joubert A. et al. “Irradiation in the presence of iodinated contrast agent results in radiosensitization of endothelial cells: consequences for computed tomography therapy”, International Journal of Radiation: Oncology Biology Physics, 62(5) p 1486-1496 (2005).

However, as for all other stress factors, the history of toxic and carcinogenic risk assessment has demonstrated that molecular and cellular models of the response to stress must be validated and parameters clearly identified. It also appears to be clear that the individual factor represents a major factor to be taken into account but the relevance of the models in question arises once again (Dorr and Hendry “Consequential late effects in normal tissues” Radiother Oncol. 61(3):223-31 (2001); Granzotto et el, “Individual susceptibility to radiosensitivity and to genomic instability: its impact on low-dose phenomena” Health Phys. 100(3):282 (2011)). As such, a reliable diagnosis of the risk associated with any genotoxic stress therefore requires sound pre-data obtained on a sufficient number of individuals, cell models, with justified parameters.

The current literature shows an increasing number of studies relating to the genotoxic effects of certain metals and metalloids (such as Al, Cd, U, As, Se and Sb) and the associated nanoparticulate forms thereof (Polya and Charlet, “Increasing arsenic risk?”, Nature Geoscience 2, p. 383-384 (2009); Akhter et al., “Cancer targeted metallic nanoparticle: targeting overview, recent advancement and toxicity concern”, Curr. Pharm. Des. 17(18), p. 1834-1850 (2011); Almeida et al., “In vivo biodistribution of nanoparticles”, Nanomedicine (Lond) 6(5), p. 815-835 (2011); Pereira et al., “Genotoxicity of uranium contamination in embryonic zebrafish cells”, Aquatic Toxicology 109, p. 11-16 (2012); Pereira et al., “Comparative genotoxicity of aluminium and cadmium in embryonic zebrafish cells”, Mutation Research 750, p. 19-26 (2013)), particularly relating to exposure via water and generated by the semiconductor industry, as well as pesticides (Garaj-Vrhovac and Zeljezic, “Evaluation of DNA damage in workers occupationally exposed to pesticides using single-cell gel electrophoresis (SCGE) assay. Pesticide genotoxicity revealed by comet assay”, Mutation Research 469(2), p. 279-285 (2000); Garaj-Vrhovac et al., “Efficacity of HUMN criteria for scoring the micronucleus assay in human lymphocytes exposed to a low concentration of p,p′-DDT”, Braz J Med Biol Res 41(6), p. 473-376 (2008); Guilherme et al., “Differential genotoxicity of Roundup® formulation and its constituents in blood cells of fish (Anguilla Anguilla): considerations on chemical interactions and DNA damaging mechanisms”, Ecotoxicology 21(5), p 1381-90. (2012)). However, these two types of agents clearly represent major societal challenges in view of industrial development, atmospheric pollution and the need to increase agricultural yields. In fact, they also find themselves at the center of health concerns both in an environmental context and in an occupational context. The scientific community suspects that the toxicity of certain oxidized nanoparticulate compounds could give rise to genotoxicity and carcinogenesis: research on the specific biological effects of nanoparticles is therefore naturally in line with the planning of research on the human and environmental effects of such technology.

It is also known that the issue of tissue sensitivity to ionizing radiation is inseparable from those of DNA damage repair mechanisms. Indeed, at a cellular level, ionizing radiation may break certain types of chemical bonds generating free radicals (in particular by peroxidation) and other reactive species causing DNA damage. The damage of DNA by endogenous or exogenous attacks (such as ionizing radiation and free radicals) may result in different types of DNA damage according to the energy deposited in particular: base damage, single-strand breaks and double-strand breaks (DSBs). Non-repaired DSBs are associated with cell death, toxicity and more specifically radiosensitivity (in the case of exposure to ionizing radiation). Poorly repaired DSBs are associated with genomic instability, mutagenic phenomena and predisposition to cancer. The body has specific repair systems for each type of DNA damage. In respect of DSB, mammals have two main repair modes: suture repair (strand ligation) and recombination repair (insertion of a homologous or non-homologous strand).

It is also known that tissue sensitivity to ionizing radiation is very variable from one organ to another and from one individual to another; the idea of “intrinsic radiosensitivity” was conceptualized by Fertil and Malaise (“Inherent cellular radiosensibility as a basic concept for human tumor radiotherapy”, Int. J. Radiation Oncology Biol. Phys. 7, p. 621-629 (1981); “Intrinsic radiosensitivity of human cell lines is correlated with radioresponsiveness of human tumors: Analysis of 101 published survival curves”, Int. J. Radiation Oncology Biol. Phys. 11, p. 1699-1707 (1985)). As such, the various studies on the therapeutic effects and side-effects of radiotherapy have demonstrated that there are individuals who exhibit particularly high radioresistance, and individuals displaying, on the other hand, radiosensitivity that may range from a clinically recognized but inconsequential side-effect to a lethal effect. Even outside of certain rare cases of extreme radiosensitivity, which appears to be of proven genetic origin, radiosensitivity is thought to stem generally from a genetic predisposition: it is therefore specific to an individual.

That which is true for radiosensitivity is also true for predisposition to cancer, and more particularly to radiation-induced cancer. As such, any excess biological dose increases both the toxic risk and the carcinogenic risk. It would therefore be useful to avail of a predictive test method to be able to determine the risk and the excess biological dose due to exposure to DNA-breaking genotoxic agents.

In the context of their publications (Joubert et al., “DNA double-strand break repair defects in syndromes associated with acute radiation response; At least two different assays to predict intrinsic radiosensitivity?”, published in Int. J. Radiation Biology 84(2), p. 107-125 (2008)), a classification of human radiosensitivity into 3 groups was proposed: Group I: radioresistance and low cancer risk, Group II moderate radiosensitivity and high cancer risk; Group III, hyper-radiosensitivity and high cancer risk. This classification is based on molecular criteria; it makes it possible to describe all cases of human radiosensitivity. Such a classification accounting for the individual factor does not exist for the genotoxic stress induced by chemical agents such as metals and pesticides.

A number of documents describe the conditions wherein H2AX or pH2AX is used as a marker of DNA damage detection and repair particularly in the case of the use of breaking agents.

The patent application WO 2014/152873 describes a method for the quantification of the genotoxicity of active substances used in chemotherapy by the quantification of histone H2AX expression.

The patent application WO 2005/113821 describes the use of the marker pH2AX as means for detecting DNA double-strand breaks in methods for identifying the least toxic tobacco products. In these methods, tobacco smoke is placed in contact with the cells for a predetermined time (15 minutes, 20 minutes, 30 minutes, 40 minutes or one hour). The presence or absence of pH2AX foci is verified by immunofluorescence. However, this method relates to a cocktail of chemical products in which the breaking agent is not known precisely.

The patent application WO2005/113 821 (Vector Tobacco/New York Medical University) describes the use of the marker pH2AX for detecting DNA double-strand breaks and evaluating tobacco toxicity. A further method which uses the level of H2AX expression for detecting DNA double-strand breaks and evaluating the efficacy of an anti-cancer agent is described in WO2014/152 873 (Pioma).

### SUMMARY

In spite of this extensive prior art, the applicant has observed that there is no method for the quantification of the excess biological dose and the risk associated with exposure to DNA-breaking chemical agents. For these agents, the problem of providing a predictive method of individual genotoxicology therefore remains without an operational solution. The present invention aims to propose a novel predictive method of the toxicity risk associated with exposure to DNA-breaking chemical agents.

The inventors observed, and the method according to the invention stems from this observation, that DNA double-strand breaks (DSBs) are the most predictive damage of genotoxicity when they are not repaired, on one hand, and of genomic instability when they are poorly repaired, on the other. Within the scope of the present invention, the inventors discovered that DSBs are handled by the majority repair mode referred to as suture, and/or by the minority defective repair mode referred to as MRE11-dependent recombination. The equilibrium between these two repair modes is controlled by the ATM protein and represents the individual factor. The marker pH2AX indicates a DSB site recognized by the suture repair mode. The marker MRE11 indicates a DSB site handled by defective MRE11-dependent repair. The marker pATM provides information on the activation of the suture pathway by phosphorylation of H2AX and inhibition of the MRE11-dependent pathway.

The inventors further observed a transfer of the cytoplasmic forms of ATM protein in the cellular nucleus following oxidative type stress, and particularly following stress inducing DSBs and producing oxidation in the cytoplasm.

The inventors demonstrated that these models are valid for a large number of chemical and biochemical DNA-breaking agents such as metals, pesticides, nanoparticles and certain chemotherapeutic drugs.

To assess the DNA damage due to an exogenous genotoxic attack, it is necessary to account for: on one hand, the spontaneous DNA state, and on the other, the stress-induced states thereof.

Moreover, after exposure to genotoxic stress, it is necessary to account for the DNA repair, the kinetics whereof is dependent on the type of stress but also potentially on the type of tissue impacted. It is further known that the efficacy and rapidity of DNA repair varies from one individual to another, and that there are furthermore specific genetic conditions leading to exceptional sensitivity.

Finally, to better ascertain between-subject differences, it is necessary to construct a system based on a biological dose or a reference concentration to better quantify the phenomena on the same basis.

According to the invention, the problem is solved by a method based on:

(i) Preparation of a cell sample by dispersion and/or amplification of non-transformed cells, sampled from a subject, for example cells from skin biopsies from the subject in question but also so-called reference control cells considered to be resistant to the breaking stress in question (e.g.: cells from Group I subjects);

(ii) Determination of a reference concentration after exposure of the cell sample from the reference cells (Group I) to the given stress. Note that this step may have already been performed and be contained in an inventing laboratory database;

(iii) Definition of a mechanistic model valid for quiescent human cells;

(iv) Functional DSB recognition, repair and signaling tests on the cells of the subject in question at the reference concentration defined above.

The so-called reference control cells are cells considered to be resistant to the breaking stress in question, preferably these are cells resistant to the stress induced by chemical agents and to radiation (e.g.: cells from Group I subject). It is possible to use commercial cells routinely used as controls in genotoxicity studies such as, in particular, the cell lines 1BR3 (Killalea et al., “Factors in post dialysis CAPD fluid affecting 3H cholesterol efflux from human skin fibroblasts”, Biochemical Society Transactions 25 p 123S (1997)), 149BR and MRC9 (Watanabe et al., “Comparison of lung cancer cell lines representing four histopathological subtypes with gene expression profiling using quantitative real-time PCR”, Cancer Cell International 10(2) p 1-12 (2010)). Further cells such as HF19, IMR90, 48BR, 70BR, 142BR, 155BR, and MRC5 may be used as so-called reference control cells.

A first aim of the invention is therefore a process for predicting the sensitivity of a subject with respect to a DNA-breaking stress using a cell sample obtained from cells (preferably skin cells) sampled on the subject and the definition of a reference concentration at which the experiments are conducted.

in which process:

(So-Called Reference Definition Step)

(i) if the reference concentration has not previously been determined by the chemical or biochemical agent to be studied, a cell sample is prepared by dispersion and/or amplification of so-called reference cells (sensitivity group I); a plurality of concentrations within a broad concentration range of said breaking agent (said concentration range ranging for example from nM to mM) is applied for a predetermined period of time (preferably 24 hours) on this cell sample; pH2AX immunofluorescence is performed with DAPI counterstaining which also enables on the same cell sample the analysis of micronuclei for all the concentrations applied.

(ii) Either the mean number of nuclear foci observed with the marker pH2AX at the observation times t and at the concentration C (this mean number being referred to as NpH2AX(t, C)), or the mean number of micronuclei per 100 cells at the observation times t and at the concentration C (this mean number being referred to as NMN(t, C)), or the standard error a corresponding to the error committed on these respective measurements which must be performed on at least 50 nuclei once (Gaussian standard error) or 3 independent experiments of 50 nuclei (standard error of the mean).

(iii) The so-called reference concentration Cref is the concentration giving:

NpH2AX(24 h,Cref)+2σ=2 or indeed NMN(24 h,Cref)+2σ=2%;

(So-Called Risk Assessment Step)

(iv) A cell sample is prepared by dispersion and/or amplification of cells sampled from the subject in question. To this cell sample is applied the reference concentration Cref defined above for a predetermined time (preferably 24 hours). A determination of pH2AX immunofluorescence is then performed with DAPI counterstaining.

(v) On said cell sample, NpH2AX(24 h, Cref) and NMN(24 h, Cref) are then determined.

(vi) If for the cell sample NpH2AX(24 h, Cref) [symbol] 2 or NMN(24 h, Cref) [symbol] 2%, then the genotoxic risk is considered to be low and described as “Group I”

(vii) If for the cell sample NpH2AX(24 h, Cref)>8 or NMN(24 h, Cref)>10%, then the genotoxic risk is considered to be very high and described as “Group III”

(viii) For all other cases, the genotoxic risk is considered to be intermediate and described as “Group II”.

A further aim of the invention is a process for evaluating the sensitivity of a tissue sampled from a subject to the DNA-breaking toxic effect of at least one chemical or biochemical agent, or of a combination of chemical and/or biochemical agents, comprising the following steps:

(a) A working concentration is set for said at least one chemical or biochemical agent, or for chemical and/or biochemical agents included in said combination of chemical and/or biochemical agents;

(b) Cells are sampled from a tissue to be evaluated of a subject;

(c) Said cells are dispersed and/or amplified so as to obtain a cell sample;

(d) Said cell sample is brought into contact with said at least one chemical or biochemical agent (or said combination and/or biochemical agents) in the working concentration thereof defined in step (a), for a predetermined period of time;

(e) The number of DNA double-strand breaks, and/or a biomarker representing this number, and/or the number of micronuclei is detected,

in the knowledge that steps (b), (c), (d) and (e) must be carried out one after the other, and that step (a) must be carried out before step (e).

Said chemical agent may be, by way of example, a metallic or non-metallic anion, a non-metallic cation, an organic anion, an organic cation, a zwitterionic compound, an optionally neutral inorganic compound, an optionally neutral organic compound, an organometallic compound, an insoluble compound; said chemical may be present for example in dissolved form in a liquid (aqueous or non-aqueous) medium, in particle form, in nanoparticle form, fixed on a cell membrane, in gaseous form.

Said biochemical agent may be, by way of example, a peptide (optionally recombinant), an antibody, an antigen, a virus (optionally deactivated), a virus fragment, a cell fragment.

Advantageously, the process according to the invention further comprises a step (f) wherein a diagnostic score is determined which represents said sensitivity of said tissue to the DNA-breaking toxic effect of said chemical or biochemical agent or of said combination of chemical and/or biochemical agents, using said number of DNA double-strand breaks (and/or the number of micronuclei) and said working concentration.

According to the invention, the detection of double-strand breaks in step (e) is carried out advantageously using a technique selected in the group formed by immunofluorescence, cytogenetic testing, pulsed-field electrophoresis.

In one embodiment, in step (e), a biomarker selected in the group formed by: pH2AX, 53BP1, Phospho-DNAPK, MDC1 is detected. Advantageously, the biomarker pH2AX is detected, and preferably the number and size of the nuclear foci of said biomarker. In one particularly preferred embodiment, counterstaining suitable for locating the cell nuclei is performed to quantify the micronuclei (MN).

In step (e) of the process according to the invention, the working concentration is advantageously a previously determined reference concentration Cref. In one embodiment, the number of DNA double-strand breaks is determined by pH2AX immunofluorescence, and, after DAPI counterstaining, the number of micronuclei (MN) is detected, and then NpH2AX(24 h, Cref) and NMN(24 h, Cref) are determined on said cell sample; if for the cell sample NpH2AX(24 h, Cref) [symbol] 2 or NMN(24 h, Cref) [symbol] 2%, then the genotoxic risk is considered to be low and/or described as “Group I”; if for the cell sample NpH2AX(24 h, Cref)>8 or NMN(24 h, Cref)>10%, then the genotoxic risk is considered to be very high and/or described as “Group III”; for all other cases, the genotoxic risk is considered to be intermediate and/or described as “Group II”.

In step (e) of the process according to the invention, the working concentration is advantageously a previously determined reference concentration Cref. This determination is performed advantageously by means of a process wherein:

(i) a cell sample is prepared by dispersion and/or amplification of so-called reference cells (sensitivity Group I) and is subdivided into a plurality of fractions;

(ii) a plurality of concentrations of said at least one chemical or biochemical agent under test is applied, said concentrations being chosen within a concentration range of said chemical or biochemical agent (said concentration range ranging for example from nM to mM) for a predetermined period of time (preferably 24 hours), in the knowledge that each on a fraction of this cell sample;

(iii) for each of the fractions of the cell sample, the number of pH2AX foci per cell and/or the number of micronuclei per cell is/are determined;

(iv) Determination is performed of:
the mean number of nuclear foci obtained with the marker pH2AX at the observation times t and at the concentration C (this mean number being referred to as NpH2AX(t, C)), (this determination being carried out preferably by pH2AX immunofluorescence with DAPI counterstaining),
the mean number of micronuclei per 100 cells at the observation times t and at the concentration C (this mean number being referred to as NMN(t, C)),
the standard error a on these respective measurements, in the knowledge that these measurements are carried out preferably on at least 50 nuclei once (Gaussian standard error) or on 3 independent experiments of 50 nuclei (standard error of the mean),
the so-called reference concentration Cref as the concentration giving:

NpH2AX(24 h,Cref)+2σ=2 or indeed NMN(24 h,Cref)+2σ=2%.

Advantageously, the so-called reference cells are chosen from the cell lines HF19, IMR90, 48BR, 70BR, 142BR, 155BR, and MRC5, 1BR3, 149BR and MRC9 and more particularly from the cell lines 1BR3, 149BR and MRC9.

In one embodiment of the process for evaluating the sensitivity of a tissue sampled from a subject to the DNA-breaking toxic effect of at least one chemical or biochemical agent, or of a combination of chemical and/or biochemical agents:

(i) cells from said sampled tissue are isolated and/or amplified, these amplified cells constituting “the cell sample”;

(ii) on said cell sample, the mean number of nuclear foci obtained with the marker pH2AX is determined at the observation times t (these mean numbers being referred to respectively as NpH2AX(t) said observation times t being the time t=0 min (referred to as t0, the non-exposed state to said at least one chemical or biochemical agent (or said combination of chemical and/or biochemical agents) and at least one observation time t4 after contacting said cell sample with said at least one chemical or biochemical agent (or said combination of chemical and/or biochemical agents) in the working concentration thereof for a predetermined period of time (this contacting being referred to herein as “genotoxic exposure”);

(iii) the sensitivity group of the sample to genotoxic exposure is determined, using at least the mean numbers NpH2AX(t);

t4 is a fixed value which represents the time for which the level of DNA breaks attains the residual value thereof, and which must be at least 12 hours, and preferably between 12 hrs and 48 hrs, and which is more preferentially approximately 24 hours;

In one embodiment, on said cell sample, the mean number of micronuclei observed at the times t per 100 cells [as a %] is determined (this mean number being referred to as NMN(t)), the time t being at least t0 (not exposed to an absorbed biological dose D) and t4 after exposure with an absorbed biological dose D.

Within the scope of the present invention, the Group criterion may be defined according to the clinical criteria: Group I=absence of clinical signs; Group II=presence of clinical signs; Group III=lethal effect.

### DRAWINGS

FIG. 1 shows the variations (A), (B), and (C) of the number of pH2AX foci 24 hours after contacting the cell samples with glyphosate (CAS No. 1071-83-6) at a given concentration according to this glyphosate concentration for the fibroblast lines 1BR3 (FIG. 1 (A)), 149BR (FIG. 1 (B)) or 04PSL (FIG. 1 (C)).

FIG. 2 shows the variations (A), (B) and (C) of the number of pH2AX foci 24 hours after contacting the cell sample with 5FU at a given concentration according to this 5FU concentration for the fibroblast lines MRC9 (FIG. 2 (A)), 03HLS (FIG. 2 (B)) and GM02718 (FIG. 2 (C)).

### DESCRIPTION

An embodiment with a plurality of alternative embodiments suitable for a human patient is described herein.

Test Preparation

Before sampling any cells and before handling any sampled cells, the respective operators (belonging for example to a cytological analysis laboratory) are informed (typically by the physician) of the patient's potential HIV or hepatitis C infection status so that said operators can take suitable increased biological safety measures when sampling, handling and managing the cell culture.

Then, the operator takes a tissue sample used for preparing the cell sample from the patient. Preferably, a skin sample is taken by biopsy; this sample may be advantageously carried out according to a method known as “skin punch” biopsy. The tissue sample is placed in DMEM medium+20% (sterile fetal calf serum). The tissue sample is transferred without delay to a specialized laboratory, in the knowledge that the sample must not remain more than 38 hours at ambient temperature.

The following step represents the isolation and/or amplification of the sampled tissue.

In one embodiment, on receipt, the tissue sample (typically the biopsy) is established in the form of an amplifiable cell line without a viral or chemical transformation agent according to an ancillary procedure well known to culture laboratories, as underlined by the publication of Elkind et al. “The radiobiology of cultured mammalian cell”, Gordon and Breach (1967). Once the number of cells is sufficient (typically after 1 to 3 weeks), the first experiments are carried out using the process according to the invention. A cell sample is prepared: The cells are inoculated on glass coverslips in Petri dishes. A portion of these coverslips are contaminated with metals or pesticides or any other DNA-breaking chemical or biochemical agent at different concentrations. A further portion is not contaminated; it represents the spontaneous state. During contamination, the cells remain in the culture incubator at 37° C.

For the contaminated cells, characteristics are acquired corresponding to the state after an incubation time with the DNA-breaking chemical or biochemical agent. Said characteristics are represented by foci corresponding to the marker pH2AX. The cells on glass coverslips are then fixed, lysed and hybridized. The following procedure, known per se (Bodgi et al, “A single formula to describe radiation-induced protein relocalization: towards a mathematical definition of individual radiosensitivity”, J Theor Biol. 21 p 333:135-45.2013):

the cells are fixed in 3% paraformaldehyde and 2% sucrose for 15 minutes at ambient temperature and permeabilized in 20 mM HEPES buffer solution (4-(2-hydroxyethyl)-1-piperazine ethane sulfonic acid) at pH 7.4, 50 mM NaCl, 3 mM MgCl2, 300 mM sucrose, 0.5% Triton X-100 (a non-ionic surfactant having the formula t-Oct-C6H4-(OCH2CH2)×OH where x=9-10, CAS No. 9002-93-1, supplied for example by Sigma Aldrich) for 3 minutes. The glass coverslips are then washed in phosphate buffer saline (known as the acronym PBS) before immunological staining. The incubation took place for 40 min at 37° C. in PBS supplemented with 2% bovine serum albumin (known as the acronym BSA or fraction V, supplied for example by Sigma Aldrich) and was followed by a wash with PBS. Anti-pH2AX primary antibodies are used at a concentration of 1:800. The incubations with anti-mouse FITC or anti-rabbit TRITC secondary antibodies (1:100, supplied by Sigma Aldrich) were performed at 37° C. in 2% BSA for 20 minutes. Glass coverslips were treated with Vectashield™ containing DAPI (4,6-Diamidino-2-phenylindole) to label the nucleus. Staining with DAPI also makes it possible, indirectly, to determine the number of cells in phase G1 (nuclei with homogenous DAPI staining), in phase S (nuclei with numerous pH2AX foci), in phase G2 (nuclei with heterogeneous DAPI staining) and metaphases (visible chromosomes).

The results are acquired using these coverslips on an immunofluorescence microscope (Olympus model for example). The reading may be direct (typically by counting the foci on at least 50 cells in G0/G1 for each point) or using dedicated image analysis software, or on an automated microscope; preferably the software or automated microscope methods are calibrated with manual determinations.

In order to obtain results of sufficient statistical reliability to serve as a basis for diagnosis, at least 3 independent series of experiments (radiation) are performed and the mean of each of the numbers of foci for the times defined is calculated.

Determination of Biological and Clinical Parameters

General and Markers Used

The invention is based, inter alia, on the use of data acquired for one of the two markers pH2AX on non-contaminated (spontaneous state) and contaminated cells. The method is based on the study of the labeling with this marker for a given contamination time: the samples are labeled after a predetermined time interval from discontinuing contamination, and the immunofluorescence thereof is studied.

The means obtained for each point and each dose with each marker are calculated with the standard errors of the mean (referred to as “SEM”) given that the sampling is n=3 (no Gaussian type “standard error SE”).

pH2AX denotes the phosphorylated forms in serine 439 of variant X of histone H2AX marking, according to the applicant's observations, the number of DNA double-strand breaks (DSBs) that are recognized by the main and faithful repair mode, suture. The marker pH2AX is essentially nuclear in the form of nuclear foci only and only the number and size of the foci shall be analyzed.

Counterstaining with DAPI (a DNA marker known to those skilled in the art) makes it possible to locate the nucleus to situate the cytoplasmic or nuclear location to quantify the micronuclei, which are complementary cell markers to the data on the foci.

Biological and Clinical Parameters

The definition and determination are performed as indicated of:
NpH2AX(t), the mean numbers of nuclear foci obtained with the markers pH2AX at the observation times t0 (non-contaminated) and t4, in the knowledge that the determination of the parameter NpH2AX(t) is mandatory within the scope of the method according to the invention,
NMN(t) the number of micronuclei observed spontaneously (at t=t0, i.e. without contamination) or at t=t4 after contamination per 100 cells (as a %).

The process according to the invention demonstrates that the tissue sensitivity to a given metal varies according to the tissue of interest. For example, astrocytes contaminated with 100 μM of aluminum exhibit less breaks (HA cells, 2 H2AX foci) compared to endothelial cells for the same concentration (HMEC cells, 3.7 H2AX foci) (see table 1). Furthermore, for some metals, the inventors demonstrated that there was a correspondence between the single toxicity scale proposed and certain clinical signs described for example in the case of lead (saturnism) or in the case of cadmium (Itai-Itai disease) (see table 3).

The process according to the invention makes it possible to also demonstrate that cells contaminated with copper exhibited for the highest concentration tested (1 mM) a number of DNA breaks visualized by H2AX foci ranging from 2 to 21 foci according to the cell type tested (see tables 1 and 2).

It is noted that the process according to the invention is so sensitive that it makes it possible to characterize the impact on a tissue of DNA-breaking chemical agents in very low concentrations, which are of the order of magnitude of the regulatory limit values for certain chemical agents in drinking water; these limit values are for example of the order of 2 mM (2 mg/L) for copper, 200 μm for aluminum, 5 μm for cadmium, 10 μM for Pb.

Predictive Evaluation

This targets the prediction of clinical parameters using the biological data measured.

A quantitative diagnosis directly derived from the mathematical value of the scores or mathematical formulas correlating the scores; this relates to the following criterion:

(i) Patient classification in a Group I, II or III (criterion referred to as GROUP):

The definition of the sensitivity groups (GROUP) helps the physician determine based on the scores according to the invention and the clinical profile of the patient analogies with known genetic syndromes. These groups were initially defined in the publication by Joubert et al. (Int. J. Radiat. Biol. 84(2), p. 107-125 (2008), cited above.

According to the present invention, it is considered that:

If for the cell sample NpH2AX(24 h, Cref)<=2 or NMN(24 h, Cref)<=0.5%, preferably NMN(24 h, Cref)<=1% or even more preferentially NMN(24 h, Cref)<=2%, then the genotoxic risk is considered to be low or described as “Group I”

If for the cell sample NpH2AX(24 h, Cref)>8 or NMN(24 h, Cref)>10%, then the genotoxic risk is considered to be very high and described as “Group III”

For all other cases, the genotoxic risk is considered to be intermediate and described as “Group II”.

### EXAMPLES

### Example 1

Determination, on Control Fibroblast Lines, of the Reference Concentration of Glyphosate (Chemical Agent)

Commercial 1BR3 and 149 BR control fibroblasts were amplified according to the recommendations of the supplier (SIGMA-ALDRICH) until the number of cells sought was obtained. After obtaining a sufficient number of cells (generally after one to 3 weeks), the first experiments were conducted using the process according to the invention. The cells were inoculated on glass coverslips in Petri dishes. A portion of these coverslips was then contacted with the medium under test comprising glyphosate (CAS No. 1071-83-6) at a given concentration presented in table 1 hereinafter.

Table 1 presents detection of the number of pH2AX foci 24 hours after contacting 1BR3, 149 BR control fibroblast cells and 04PSL cells with glyphosate according to the glyphosate concentration used.

TABLE 1

| glyphosate | 1BR3 (control cells) | | | 149BR (control cells) | | | 04PSL | | |
|---|---|---|---|---|---|---|---|---|---|
| concentration | | | pH2AX(24) + | | | pH2AX(24) + | | | pH2AX(24) + |
| (μM) | pH2AX(24) | SEM | 2xSEM | pH2AX(24) | SEM | 2xSEM | pH2AX(24 h) | SEM | 2xSEM |
| 3 | 1.6 | 0.128 | 1.856 | 0.8 | 0.064 | 0.928 | 1.7 | 0.136 | 1.972 |
| 10 | 1.6 | 0.128 | 1.856 | 1 | 0.08 | 1.16 | 1.8 | 0.144 | 2.088 |
| 30 | 1.9 | 0.152 | 2.204 | 1.5 | 0.12 | 1.74 | 4.1 | 0.328 | 4.756 |
| 100 | 2 | 0.16 | 2.32 | 1.8 | 0.144 | 2.088 | 6 | 0.48 | 6.96 |
| 300 | 2.3 | 0.184 | 2.668 | 3 | 0.24 | 3.48 | 8.4 | 0.672 | 9.744 |

After contacting with glyphosate at a given concentration, the cells were stored in the culture incubator at 37° C. 24 hours after contacting with glyphosate at a given concentration as presented in table 1, the mean number of nuclear foci obtained with the marker pH2AX was acquired. The acquisition of the results was carried out using these coverslips on an immunofluorescence microscope (Olympus model). The reading was performed directly by counting the foci obtained with the marker pH2AX on at least 50 cells in G0/G1 for each point or using dedicated image analysis software (imageJ).

In order to obtain results of sufficient statistical reliability to serve as a basis for diagnosis, 3 independent series of experiments were performed. The mean and standard errors of the mean (“SEM” or σ) of each of the numbers of foci acquired after 24 hours of contacting the control cells with glyphosate at a given concentration was calculated and presented in table 1.

As such, for the skin control cell samples 1BR3 and 149BR (see table 1), the reference concentration was determined. This reference concentration was defined as being the concentration giving: NpH2AX(24 h, Cref)+2σ=2

where σ corresponds to the standard error of the measurements of the number of pH2AX foci acquired 24 hours after contacting the control cells with glyphosate at a given concentration, these measurements being carried out on 3 independent experiments of 50 cells (standard error of the mean).

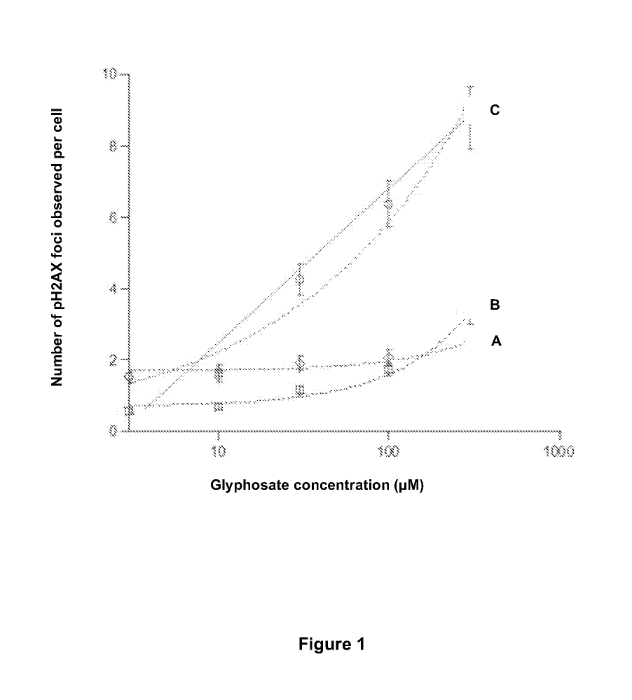

FIG. 1 represents the variation of the number of pH2AX foci acquired per cell, 24 hours after contacting the control cells 1BR3 (see FIG. 1 (A)) and 149 BR (see FIG. 1 (B)) with glyphosate according to the glyphosate concentration used. The concentration Cref defined by NpH2AX(24 h, Cref)+2σ=2 for the 2 control cells lines 149BR and 1BR3 is 100 μM.

Test Preparation (Cell Lines 04PSL)

A skin cell sample from a patient was sampled by biopsy via the “skin punch” method known to those skilled in the art. The cell sample was then placed in DMEM medium+20% sterile fetal calf serum. The cell sample was then transferred without delay to a specialized laboratory, so that the sample remained not more than 38 hours at ambient temperature.

On receipt, the cell sample from the biopsy was established in the form of an amplifiable 04PSL cell line according to a procedure well known to culture laboratories and those skilled in the art: using particularly the trypsin dispersion, the cells are once again diluted in replenished medium and so on until the number of cells sought is obtained. After obtaining a sufficient number of cells (generally after one to 3 weeks), the first experiments were conducted using the process according to the invention. The 04PSL line cells were inoculated on glass coverslips in Petri dishes. A portion of these coverslips was then contacted with glyphosate at a concentration of 100 μM. By way of verification, a further portion of these coverslips was contacted with glyphosate at a given concentration (see table 1, FIG. 1 (C)).

After contacting with glyphosate at a given concentration, the cells were stored in the culture incubator at 37° C. 24 hours after contacting with glyphosate at a given concentration as presented in table 1, the mean number of nuclear foci obtained with the marker pH2AX was acquired. The acquisition of the results was carried out using these coverslips on an immunofluorescence microscope (Olympus model). The reading was performed directly by counting the foci obtained with the marker pH2AX on at least 50 cells in G0/G1 for each point or using dedicated image analysis software (imageJ).

In order to obtain results of sufficient statistical reliability to serve as a basis for diagnosis, 3 independent series of experiments were performed. The mean and standard errors of the mean (“SEM” or σ) of each of the numbers of foci acquired after 24 hours of contacting the control cells with glyphosate at a given concentration was calculated and presented in table 1 and in FIG. 1 (C).

Determination of Genotoxic Risk of Cell Line 04PSL

At a glyphosate concentration of 100 μM, the number of pH2AX foci obtained for the cell line 04PSL is approximately 7; this figure validates the equation 2<NpH2AX(24 h)<8. Consequently, for the cell line 04PSL, the genotoxic risk associated with glyphosate is “group II” or described as intermediate. The line 04PSL is chemosensitive.

### Example 2

Determination, on Control Fibroblast Lines, of the Reference Concentration of the Chemotherapeutic Drug 5FU (Chemical Agent)

Commercial MRC9 control fibroblasts were amplified according to the recommendations of the supplier (SIGMA-ALDRICH) until the number of cells sought was obtained. After obtaining a sufficient number of cells (generally after one to 3 weeks), the first experiments were conducted using the process according to the invention. The cells were inoculated on glass coverslips in Petri dishes. A portion of these coverslips was then contacted with the medium under test comprising 5FU at a given concentration presented in table 2 hereinafter.

Table 2 presents a detection of the number of pH2AX foci 24 hours after contacting MRC9 control fibroblast cells and GM02718 and 03HLS cells with 5FU according to the 5FU concentration used

TABLE 2

| 5FU | MRC9 (control cells) | | | GM002718 | | | 03HLS | | |
|---|---|---|---|---|---|---|---|---|---|
| concentration | | | pH2AX(24 h) + | | | pH2AX(24 h) + | | | pH2AX(24 h) + |
| (μM) | pH2AX(24 h) | SEM | 2xSEM | pH2AX(24 h) | SEM | 2xSEM | pH2AX(24 h) | SEM | 2xSEM |
| 0.00 | 0.00 | 0.00 | 0.00 | 0.00 | 0.00 | 0.00 | 0.00 | 0.00 | 0.00 |
| 1.00 | 1.16 | 0.41 | 1.97 | 2.70 | 0.31 | 3.32 | 1.40 | 0.04 | 1.49 |
| 3.00 | 1.38 | 0.28 | 1.93 | 2.41 | 0.01 | 2.43 | 1.69 | 0.29 | 2.26 |
| 10.00 | 0.30 | 0.11 | 0.51 | 2.96 | 0.46 | 3.87 | 1.63 | 0.03 | 1.69 |
| 30.00 | 1.18 | 0.41 | 2.01 | 2.38 | 0.00 | 2.38 | 2.00 | 0.29 | 2.59 |

After contacting with 5FU at a given concentration, the cells were stored in the culture incubator at 37° C. 24 hours after contacting with 5FU at a given concentration as presented in table 2, the mean number of nuclear foci obtained with the marker pH2AX was acquired. The acquisition of the results was carried out using these coverslips on an immunofluorescence microscope (Olympus model). The reading was performed directly by counting the foci obtained with the marker pH2AX on at least 50 cells in G0/G1 for each point or using dedicated image analysis software (imageJ).

In order to obtain results of sufficient statistical reliability to serve as a basis for diagnosis, 3 independent series of experiments were performed. The mean and standard errors of the mean (“SEM” or σ) of each of the numbers of foci acquired after 24 hours of contacting the control cells with 5FU at a given concentration was calculated and presented in table 2.

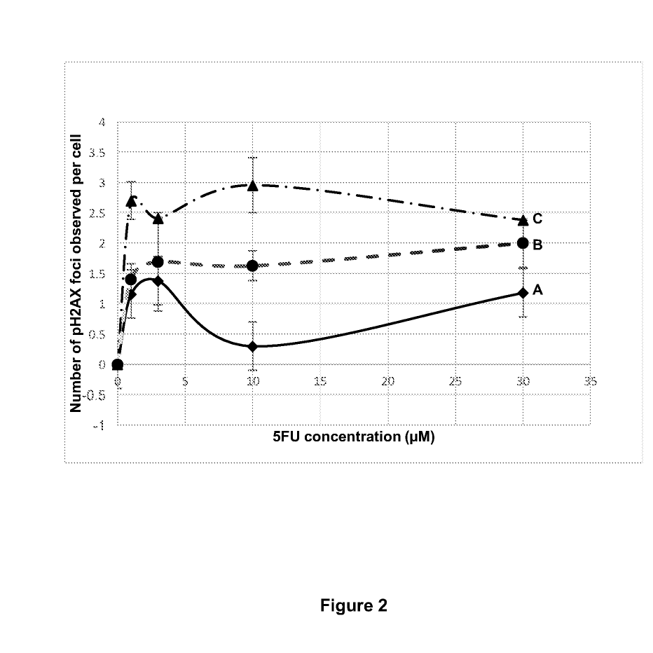

As such, for the skin control cell samples MRC9 (see table 2, FIG. 2 (A)), the reference concentration was determined. This reference concentration was defined as being the concentration giving: NpH2AX(24 h, Cref)+2σ=2

where σ corresponds to the standard error of the measurements of the number of pH2AX foci acquired 24 hours after contacting the control cells with glyphosate at a given concentration, these measurements being carried out on 3 independent experiments of 50 cells (standard error of the mean).

FIG. 2 represents the variation of the number of pH2AX foci acquired per cell, 24 hours after contacting the control cells MRC9 (see FIG. 2 (A)) with 5FU according to the 5FU concentration used. The concentration Cref defined by NpH2AX(24 h, Cref)+2σ=2 for the control cells line MRC9 is 30 μM.

Test Preparation (Cell Lines GM02718 and 03HLS)

The cell line GM02718 was amplified according to the recommendations of the supplier (Coriell Institute) until the number of cells sought was obtained.

For the line 03 HLS, a skin cell sample from a patient was sampled by biopsy via the “skin punch” method known to those skilled in the art. The cell sample was then placed in DMEM medium+20% sterile fetal calf serum. The cell sample was then transferred without delay to a specialized laboratory, so that the sample remained not more than 38 hours at ambient temperature.

On receipt, the cell sample from the biopsy was established in the form of an amplifiable 03HLS cell line according to a procedure well known to culture laboratories and those skilled in the art: using particularly the trypsin dispersion, the cells are once again diluted in replenished medium and so on until the number of cells sought is obtained.

After obtaining a sufficient number of cells (generally after one to 3 weeks), the first experiments were conducted using the process according to the invention. The GM02718, or 03HLS, line cells were inoculated on glass coverslips in Petri dishes. A portion of these coverslips was then contacted with 5FU at a concentration of 30 μM. By way of verification, a further portion of these coverslips was contacted with 5FU at a given concentration (see table 2, see FIG. 2 (B) for the cell line GM02718, respectively FIG. 2 (C) for the cell line 03HLS).

After contacting with 5FU at a given concentration, the cells were stored in the culture incubator at 37° C. 24 hours after contacting with 5FU at a given concentration as presented in table 2, the mean number of nuclear foci obtained with the marker pH2AX was acquired. The acquisition of the results was carried out using these coverslips on an immunofluorescence microscope (Olympus model). The reading was performed directly by counting the foci obtained with the marker pH2AX on at least 50 cells in G0/G1 for each point or using dedicated image analysis software (imageJ).

In order to obtain results of sufficient statistical reliability to serve as a basis for diagnosis, 3 independent series of experiments were performed. The mean and standard errors of the mean (“SEM” or σ) of each of the numbers of foci acquired after 24 hours of contacting the control cells with 5FU at a given concentration was calculated and presented in table 2 and in FIG. 2 (B) for the cell line GM02718, respectively FIG. 2 (C) for the cell line 03HLS.

Determination of the Genotoxic Risk of the Cell Line GM02718, or 03HLS Respectively

At a 5FU concentration of 30 μM, the number of pH2AX foci obtained for the cell line GM02718, or 03HLS respectively, is approximately 2.38 or 2.59 foci respectively; this figure validates the equation 2<NpH2AX(24 h)<8. Consequently, for the cell line GM02718, or 03HLS respectively, the genotoxic risk associated with 5FU is “group II” or described as intermediate. The lines GM02718 and 03HLS are chemosensitive.

### Further Examples

The tables hereinafter summarize the results of numerous experiments which were carried out as described in the “Detailed description” section above.

In tables 3 and 4, “pH2AX” corresponds to the mean number of nuclear foci obtained with the marker pH2AX, 24 hours after contacting the cell sample with the chemical agent at the concentration C (NpH2AX(24 h, C)), and where “Micronuclei” corresponds to the mean number of micronuclei observed per 100 cells 24 hours after contacting the cell sample with the chemical agent at the concentration C(N_{MN}(24 h, C)).

Table 3 presents a detection of the number of ph2AX foci and the number of micronuclei 24 hours after contacting 04PSL, 01PAU, 08HNG, 1BR3 cells with a pesticide breaking agent (Glyphosate, Permethrin, Thiobendazole, PCP, Atrazine) according to the pesticide concentration used.

TABLE 3

| | Breaking | | Concentration [μM] | | | | | | |
|---|---|---|---|---|---|---|---|---|---|
| Line | agent | Markers | 0 | 3 | 10 | 30 | 100 | 150 | 300 |
| 04PSL | Glyphosate | micronuclei | 1 | 6 | 4 | 10 | | 12 | 14 |
| | | pH2AX | 0 | 1.7 | 1.9 | 4.8 | 6.5 | | 8.7 |

| | Breaking | | Concentration [μM] | | | | |
|---|---|---|---|---|---|---|---|
| Line | agent | Markers | 0 | 0.3 | 1 | 5 | 10 |
| 01PAU | Permethrin | micronuclei | 2 | 2 | 2 | 4 | 6 |
| | | pH2AX | 0 | 0.1 | 0.2 | 1.3 | 1.8 |
| | Breaking | | Concentration [μM] | | | | |
| Line | agent | Markers | 0 | 0.3 | 1 | 3 | 10 |
| 08HNG | Thiobendazole | micronuclei | 0 | 2 | 4 | 10 | 10 |
| | | pH2AX | 0 | 1.8 | 2 | 4.3 | 6 |

| | Breaking | | Concentration [μM] | | | | | | |
|---|---|---|---|---|---|---|---|---|---|
| Line | agent | Markers | 0 | 0.3 | 3 | 10 | 30 | 50 | 100 |
| 1BR3 | PCP | micronuclei | 1 | 2 | 4 | 10 | | 10 | 20 |
| | | pH2AX | 0 | 1.2 | | 1.5 | 1.9 | | 4.3 |

| | Breaking | | Concentration [μM] | | | | | | | |
|---|---|---|---|---|---|---|---|---|---|---|
| Line | agent | Markers | 0 | 0.01 | 0.1 | 0.3 | 1 | 10 | 20 | 30 |
| 1BR3 | Atrazine | micronuclei | 1 | 5 | 6 | | 13.5 | 15 | 40 | |
| | | pH2AX | | 1 | 1.3 | 1.8 | | 3.1 | | 3.5 |

Table 4 presents a detection of number of pH2AX foci and number of micronuclei 24 hours after contacting control nervous system cell lines Ha (astrocyte cells), Hah (hippocampus astrocyte cells) and Hasp (spinal cord astrocyte cells) with a metallic compound (AlCl₃, Cu, CuCl₂, CuSO₄, Pb(NO₃)₂, CdCl₂, Cd-acetate or Cd-acetate-citrate) according to the concentration of said metallic compound used.

TABLE 4

| | Breaking | | Concentration [μM] | | | | | |
|---|---|---|---|---|---|---|---|---|
| Line | agent | Marker | 3 | 10 | 30 | 100 | 300 | 1000 |
| Ha | AlCl₃ | pH2AX | 0.34 | 0.88 | 1.37 | 2.18 | 2.96 | 6.04 |
| | | Micronuclei | 10 | 15 | 16.7 | 46.7 | 56.7 | 70 |
| | Cu | pH2AX | | 0.3 | 0.62 | 2.55 | 0 | 0 |
| | | Micronuclei | | 4 | 5 | 13 | 20 | 50 |
| Hah | AlCl₃ | pH2AX | 0.07 | 0.68 | 0.83 | 1.22 | 1.77 | 2.28 |
| | | Micronuclei | 2 | 2 | 4 | 6 | 20 | 30 |
| | Cu | pH2AX | | 1.01 | 1.87 | 2.62 | 5.45 | 6.61 |
| | | Micronuclei | | 6 | 10.7 | 20.7 | 46.7 | 73.3 |
| Hasp | AlC₃ | pH2AX | 0.84 | 0.46 | 0.74 | 2.15 | 1.7 | 1.62 |
| | | Micronuclei | 2 | 4 | 4 | 7.3 | 8.7 | 13 |
| | Cu | pH2AX | | 0.29 | 0.64 | 1.13 | 1.78 | 2.36 |
| | | Micronuclei | | 4 | 7 | 9 | 10 | 16 |
| | AlC₃ | pH2AX | 3.2 | 3.2 | 5.7 | 3.7 | 4.5 | 9.9 |
| | | Micronuclei | 1.75 | 3.75 | 5 | 4.33 | 7.33 | 11.5 |
| | CuCl₂ | pH2AX | 1.6 | 1.9 | 1.6 | 1.5 | 5.3 | 21.4 |
| | | Micronuclei | 4 | 6.7 | 5.3 | 6.7 | 7.3 | 4 |
| | CuSO₄ | pH2AX | 1.9 | 2.3 | 3.4 | 6.4 | 20.1 | 30.2 |
| | | Micronuclei | 3 | 5 | 4.7 | 5.3 | 9.3 | 4 |
| | Pb(NO₃)₂ | pH2AX | | 4 | 7.5 | 15 | 21 | |
| | | Micronuclei | | 0 | 7 | 12 | 20 | 10 |
| | CdCl₂ | pH2AX | | 1.9 | 3.7 | 8.3 | | |
| | | Micronuclei | | 3.5 | 9 | 12.25 | | |
| | Cd- | pH2AX | | 5 | 5.5 | 7 | | |
| | acetate | Micronuclei | | 5 | 10 | 10 | | |
| | Cd- | pH2AX | | 1 | 4.75 | 7 | | |
| | acetate- | Micronuclei | | | | | | |
| | citrate | | | | | | | |

The experimental data presented in table 4 above were used to determine the reference concentration C_{ref} particularly for Pb(NO₃)₂ (C_{ref}<1 μM) and CdCl₂ (C_{ref}=10 μM). These data were correlated with the clinical signs observed and presented in table 5 hereinafter, particularly for Pb(NO₃)₂ and CdCl₂.

Table 5 presents numerical examples of correspondence between the single toxicity scale according to the invention and the corresponding clinical signs.

TABLE 5

| Chemical | Reference | Prediction based | |
|---|---|---|---|
| species | concentration C_{ref} | on algorithm | Clinical effects observed |
| Lead | <1 μM | “Group II” risk | Onset of signs of saturnism above 100 μg/l of |
| [Salt used: | N_{pH2AX}(24 h) < 2 | predicted between | blood corresponding to approximately 2 μM |
| Pb(NO₃)₂] | | 1 and 30 μM | |
| | | 2 < N_{pH2AX}(24 h) < 8 | |
| | | “Group III” risk | Immediate lethal effect never actually observed |
| | | predicted above | |
| | | 30 μM | |
| | | N_{pH2AX}(24 h) > 8 | |
| Cadmium | 10 μM | “Group II” risk | Relating to exposures sustained by some inhabitants |
| [salt used: | N_{pH2AX}(24 h) < 2 | predicted between | of the district of Toyama (Japan) following |
| CdCl₂] | | 10 and 100 μM | systemic cadmium poisoning (Itai-Itai disease) |
| | | 2 < N_{pH2AX}(24 h) < 8 | |
| | | “Group III” risk | Fatal fume concentrations: 40-50 mg/m³ i.e. 245 μM |
| | | predicted above 100 μM | per m³ (death in 100 min) |
| | | N_{pH2AX}(24 h) > 8 | |
| Chromium | 3 nM | “Group II” risk | Cases of poisoning in Hinkley USA, Erin Brockovich case) |
| [salt used: | N_{pH2AX}(24 h) < 2 | predicted between | with 1.19 g/l in well water equivalent to 4.6 mM. The |
| Na₂CrO₄] | | 3 and 30 nM | concentration in tap water was estimated at 23 Nm |
| | | 2 < N_{pH2AX}(24 h) < 8 | |
| | | “Group III” risk | |
| | | predicted above 30 nM | |
| | | N_{pH2AX}(24 h) > 8 | |

Where the GROUP criterion is defined as follows: GROUP I=absence of clinical signs, GROUP II=presence of clinical signs, and GROUP III=lethal effect.

1-15. (canceled)

16. A method for evaluating the sensitivity of a tissue sampled from a subject to a DNA-breaking toxic effect of at least one chemical agent or biochemical agent, the method comprising:
establishing a working concentration for said at least one chemical or biochemical agent, or of chemical and/or biochemical agents;
sampling, after establishing the working concentration, cells from a tissue to be evaluated of a subject;
dispersing and/or amplifying, after the sampling, said cells to obtain a cell sample;
bringing, for a predetermined period of time, and after the dispersing and/or the amplifying, said cell sample into contact with said at least one chemical agent or biochemical agent in the working concentration; and
detecting, after the bringing, a number of DNA double-strand breaks, and/or a biomarker representing said number, and/or a number of micronuclei.

17. The method of claim 16, further comprising determining a diagnostic score which represents said sensitivity of said tissue to the DNA-breaking toxic effect of said at least one chemical agent or biochemical agent, using said number of DNA double-strand breaks, and/or said number of micronuclei, and said working concentration.

18. The method of claim 16, wherein the detection is carried out using a technique selected from the group consisting of immunofluorescence, cytogenetic testing, and pulsed-field electrophoresis.

19. The method of claim 16, wherein detecting said biomarker comprises detecting a biomarker selected from the group consisting of pH2AX, 53BP1, Phospho-DNAPK, and MDC1.

20. The method of claim 16, wherein detecting said biomarker comprises detecting biomarker pH2AX, and a number and size of nuclear foci of said biomarker.

21. The method of claim 16, further comprising performing counterstaining suitable for locating the cell nuclei to quantify the micronuclei (MN).

22. The method of claim 16, wherein:
in the detecting, a working concentration is a previously determined reference concentration C_{ref},
the number of DNA double-strand breaks is determined by pH2AX immunofluorescence, and, after DAPI counterstaining, the number of micronuclei (MN) is detected, then N_{pH2AX}(24 h, C_{ref}) and N_{MN}(24 h, C_{ref}) are determined on said cell sample.

23. The method of claim 22, wherein:
it is inferred that a genotoxic risk is low and/or described as “Group I” if for the cell sample N_{pH2AX}(24 h, C_{ref})≤2 or N_{MN}(24 h, C_{ref})≤2%, and
it is inferred that the genotoxic risk is very high and/or described as “Group III” if for the cell sample N_{pH2AX}(24 h, C_{ref})>8, or N_{MN}(24 h, C_{ref})>10%,
for all the other cases, it is inferred that the genotoxic risk is intermediate and/or described as “Group II.”

24. The method of claim 22, wherein said working concentration is a previously determined reference concentration C_{ref}.

25. The method of claim 24, wherein said previously determined reference concentration C_{ref} is performed by:
preparing a cell sample by dispersion and/or amplification of reference cells (sensitivity Group I), and subdividing the cell sample into a plurality of fractions,
applying a plurality of concentrations of the at least one chemical agent or biochemical agent under test, said concentrations being chosen within a concentration range of said at least one chemical agent or biochemical agent, said concentration range between nM to mM, for a predetermined period of time,
determining, for each of fraction of the cell sample, a number of pH2AX foci per cell and/or a number of micronuclei per cell.

26. The method of claim 25, wherein the determination of, for each of fraction of the cell sample, the number of pH2AX foci per cell and/or the number of micronuclei per cell is performed by determining:
via pH2AX immunofluorescence with DAPI counterstaining, a mean number (N_{pH2AX}(t, C)) of nuclear foci obtained with the marker pH2AX at observation times t and at concentration C,
a mean number (N_{MN}(t, C)) of micronuclei per 100 cells at the observation times t and at the concentration C,
a standard error a on each respective determination, and
the reference concentration C_{ref} as a concentration with N_{pH2AX}(24 h, C_{ref})+2σ=2 or N_{MN}(24 h, C_{ref})+2σ=2%.

27. The method of claim 25, wherein the reference cells are chosen from cell lines HF19, IMR90, 48BR, 70BR, 142BR, 155BR, 1BR3, 149BR, and MRC9.

28. The method of claim 16, wherein said at least one chemical agent is chosen from the group consisting of a metallic or non-metallic anion, a non-metallic cation, an organic anion, an organic cation, a zwitterionic compound, an optionally neutral inorganic compound, an optionally neutral organic compound, an organometallic compound, and an insoluble compound.

29. The method of claim 16, wherein said at least one chemical agent or biochemical agent is in at least one of:
dissolved form in a liquid medium,
particle form,
nanoparticle form,
fixed on a cell membrane, or
in gaseous form.

30. The method of claim 16, wherein said at least one biochemical agent is chosen from the group consisting of a peptide, an antibody, an antigen, a virus, a virus fragment, and a cell fragment.

31. The method of claim 16, wherein cells from said sampled tissue are isolated and/or amplified, said amplified cells being the cell sample.

32. The method of claim 31, further comprising:
determining, on said cell sample, a mean number (N_{pH2AX}(t)) of nuclear foci obtained with a marker pH2AX at observation times between a time t0 in a non-exposed state to said at least one chemical agent or biochemical agent, and at least one observation time t4 after contacting said cell sample with said at least one chemical agent or biochemical agent for a predetermined period of time, said contacting serving as genotoxic exposure,
determining a sensitivity group of the sample to the genotoxic exposure, using at least the determined mean numbers N_{pH2AX}(t),
determining a mean number (N_{MN}(t)) of micronuclei observed at the times t per 100 cells [as a %] on said cell sample, at least at the time t0 and at the time t4.

33. The method of claim 32, wherein t4 comprises a fixed value which represents a time for which a level of DNA breaks attains a residual value thereof.

34. The method of claim 33, wherein t4 is approximately 24 hours.

35. A method for evaluating the sensitivity of a tissue sampled from a subject to a DNA-breaking toxic effect of a combination of chemical agents and/or biochemical agents, the method comprising:
establishing a working concentration for said chemical agents and/or biochemical agents included in said combination of chemical agents and/or biochemical agents;
sampling, after establishing the working concentration, cells from a tissue to be evaluated of a subject;
dispersing and/or amplifying, after the sampling, said cells to obtain a cell sample;
bringing, for a predetermined period of time, and after the dispersing and/or the amplifying, said cell sample into contact with said combination of chemical agents and/or biochemical agents in the working concentration; and
detecting, after the bringing, a number of DNA double-strand breaks, and/or a biomarker representing said number, and/or a number of micronuclei.
